(19) United States
(12) Patent Application Publication
(10) Pub. No.: US 2010/0166832 A1
(43) Pub. Date: Jul. 1, 2010

(54) Silver coated nylon fibers and associated methods of manufacture and use

(76) Inventors: Edmund Michael Ingle, Winston-Salem, NC (US); Bennett James Fisher, Morganton, NC (US); Jerry W. Finney, Hickory, NC (US)

Correspondence Address: Edmund Ingle, 129 Cascade Ave., Winston-Salem, NC, 27127, US

(21) Appl. No.: 12/317,732

(22) Filed: Dec. 29, 2008

(51) Int. Cl. A61K 9/70; B32B 27/02; B05D 3/02; A41B 9/00; A41C 3/00; A47G 9/00; A41D 1/06; A61K 33/38; B32B 27/34

(52) U.S. Cl. 424/443; 442/123; 427/314; 424/618; 2/114; 2/400; 2/228; 450/1; 5/495; 5/490

(57) ABSTRACT

Silver coated nylon fibers are disclosed that can be used to make fabrics that are silver coated on one side of the fabric. Silver coated nylon fabrics provide an antimicrobial surface which remains on the surface and retains its antimicrobial characteristic until destroyed. The methods of adherence of the silver to the nylon fabric may be performed in a number of ways. The creation of a single fabric with one side silver coated and the other side being dyed or left natural is encompassed within this invention. In addition there can be more than two yarns creating the fabric such as the use of spandex to provide a more elastic garment.

### FIELD OF THE INVENTION

The invention relates to methods of making and utilizing silver coated nylon fabrics. The invention also relates to overcoming the existing technological barriers for dyeing silver coated nylon fabrics. Prior silver coated nylon fabrics were 100% nylon and the surface was 100% silver coated following the fabric manufacturing process. Such fabrics are not suitable for end uses because of the critical obstacle of consumer desire for color in apparel. The fabrics currently available do not allow the application of color because 100% silver coated nylon fabric can not hold dyes. Conversely, this invention involves creating silver coated nylon fabrics by incorporating the silver during or after the fabric making process, thereby adding color dyes to the non silver plated fibers. Silver coated fabrics utilized in this invention use as an antimicrobial thermally conductive and electrically conductive fabric. The textile fabric, of the present invention, possesses a silver coating that remains on the surface of the fabric, retaining its characteristics for the life of the yarn. It is not subject to loss of effectiveness after a substantial number of standard washings and dryings. Accordingly, the invention encompasses silver coating of two sided, colored and multi fiber fabrics.

### DISCUSSION OF THE PRIOR ART

Silver kills hundreds of different types of bacteria and has been used for years in the medical field due to its antibacterial efficacy. There are many examples of present day medical products which utilize 100% silver coated nylon fabrics. These fabrics are used to treat burn victims and patients with open wounds to inhibit the growth of bacteria and kill any bacteria already in the wound. Silver has also been applied topically to fabrics as an antimicrobial agent but the process has lacked efficacy because the % of silver is generally less than 3%, the fabrics are not conductive because there is not a 100% coating of silver. These fabrics can be dyed but do not have the thermal conductivity or electrical conductivity of 100% silver plated fabrics. Other companies have added silver in the synthetic fiber manufacturing process, allowing the fibers to be dyed but the amount of silver in the polymer is generally less than 3%. These fibers are dyeable but are not thermally or electrically conductive and are not as effective in killing bacteria due to the low concentration of silver.

It would be extremely beneficial to generate effective permanent antimicrobial characteristics for film or textile surfaces, particularly on apparel fabrics, while maintaining the ability to add color to these fabrics.

In addition, silver-containing nylon fiber and fabrics are available commercially for electromagnetic shielding. Micro biocides have been adapted for incorporation within melt spun synthetic fibers, as taught within Japanese unexamined Patent Application No. H11-124729, which is herein incorporated by reference in it's entirety. Although these fabrics can be dyed, they still suffer from drawbacks including the inability to conduct electricity, and ineffective antimicrobial activity.

Previously, attempts to apply these specific micro-biocides on the surfaces of fabrics and yarns have been unsuccessful. The dyeable fabrics as previously manufactured had a durability issue if treatment was topical and antimicrobial efficacy drawbacks (i.e., the kill rate above 99%/log kill rate ratio is over 2.0) if silver was added to the polymer prior to spinning. The topical approach of applying silver, with or without binders or the melt spinning of polymers with silver additives dosed in prior to spinning, generated fabric with a very low percentage of silver (or other equivalent biocidal metal). Thus, using the old methodology, the efficacy is much less than the electrolessly deposited silver coated nylon.

Topical treatment with silver or other metallic substrate has been successfully applied to fabrics that without washing are somewhat durable. Although these silver-based agents provide relatively good, semi-durable, antimicrobial properties, they tend not to produce the desired long-lasting, wash-resistant, silver-based antimicrobial textile. Moreover, these fabrics tend to lack the thermal and electrical conductive properties of electrolessly silver plated fabrics. Silver coated nylon/non-nylon fabrics are desirable for textile applications, particularly if one wants the full biocidal strength of a silver coated nylon, yet needs the hand and color for normal textile uses. Such a combination fabric permits treatment of a fabric's individual fibers prior to or after, weaving and knitting, in order to provide greater versatility to the target yarn without altering its physical characteristics. Such a “combination” fabric must prove to be wash durable, particularly for apparel fabrics, in order to be functionally acceptable. Further, it is highly desirable for such a metalized treatment to be electrically conductive on the target fabric, yarn, and/or film surface. With the presence of metals and metal ions, a wash durable, antimicrobial, dyeable and electrically conductive treatment has not been previously available in the past. Such an improvement would thus provide an important advancement within the fabric and textile art. Although antimicrobial activity is one desired characteristic of the inventive metal-treated fabric, or film, this is not a required property of the inventive article. Odor-reduction, thermal conductivity, distinct colorations, reduced discolorations, improved yarn and/or fabric strength, resistance to sharp edges, etc., are all either individual or aggregate properties which may be accorded the user of such an inventive treated yarn, fabric, or film.

### DESCRIPTION OF THE INVENTION

This application discloses fabrics which selectively exhibit antimicrobial characteristics. In one aspect of the invention, the fabric is comprised of two or more fibers, one of which is silver coated, and the other which is not silver coated. These fibers can be combined such that the silver coated fiber is predominantly on one side and the other non-silver coated fibers are on the reverse.

It is an object of the invention to provide a simple and effective manner of providing a coating on one side of a textile, fabric, or film with silver (or another metal) without coating the other side of the textile, fabric or film. This will allow the other side of the textile, fabric or film to be dyed or left in its natural state. A further object of the invention is to provide a textile, fabric or film that is electrically conductive and thermally conductive on one side so as to aid in the transportation and dissipation of heat and static build up while at the same time looking and feeling like a normal textile, fabric or film on the other side.

In one embodiment, the side of the textile, fabric, or film with the silver coating is nylon. One of the benefits of the present invention is that the use of nylon (and electrolessly plating the nylon side with silver) provides a textile, fabric or film that is far more durable than the corresponding topical treatment of textiles, fabrics or films with binders (which are subsequently treated with silver or another metal). In an embodiment, after plating, the fabric is not altered in terms of drape, and the non silver plated fiber or fibers of the fabric still have the same hand before being treated

In addition, when the textile or fabric that has been electrolessly silver plated and cut and sewn into garments with the silver side of the fabric against the skin, wearers identify a positive effect on the wearer in terms of moving moisture off of the skin. This movement and evaporation of moisture from the skin is a result of the silver coated fibers reaching body temperature very rapidly due to the inherent thermal conductivity of silver. In addition, silver does not absorb moisture and unlike some fibers, such as cotton and wool, is not hydrophilic. The silver covers 100% of the surface of the nylon fibers and by accelerating the evaporation process, cools the wearer. Thus, the evaporation process is accelerated and the moisture that the wearer generates does not get absorbed by the silver coated nylon. The effect is pronounced when a two sided material such as un-plated polyester on the exterior of the garment (i.e., the side furthest away from the skin) is used. The exterior side of the fabric can aid in the wicking of the moisture across the fabric by capillary action and or surface treatments. In this embodiment, not only is there dissipation of heat from the wearer, but the wearer can safely wear an aesthetic garment that can be dyed or printed on the non silver plated side, and worn with the printed or dyed fabric away from the skin. The fabric can be constructed in a manner that disguises the fact that there is a silver coated side to the garment.

Due to the nature of electrolessly plated nylon, the fabric can be washed in accordance with wash procedure AATCC test method 130-1981 for a plurality of washings and there is no measurable change in the surface of the silver coating. This demonstrates the ability of the textile, fabric, or film to perform in the same manner as when the product was first made. In one variation, the fabric can be washed 100 times without losing its effectiveness. Accordingly, in one embodiment, this invention encompasses a textile, fabric, or film that has two or more components, with at least one of the components being made of nylon. The fabric is electrolessly plated, but only the nylon takes on the silver. The other components of the textile, fabric or film remain untreated. Thus, the textile, fabric, or film is comprised of individual fibers, with at least one of the fibers being nylon. The finish is adhered to at least one portion of the surface of the substrate and the electrolessly plated silver finish is integrally retained on the nylon surface of the substrate. Even after a plurality of washes as performed in accordance with the wash procedure of AATCC Test Method 130-1981, an amount of at least 65% of the amount of silver finish remains present on nylon portion of the surface of said substrate. The wash durability test noted above is standard and, as will be well appreciated by one of ordinary skill in this art, is not intended to be a required or limitation within this invention. Such a test method merely provides a standard which, upon 30 washes in accordance with such, the inventive treated substrate will not lose an appreciable amount of its electrically conductive metal treatment. In an embodiment, such wash durability will be maintained after 15 washes, after 20 washes, and alternatively 30 washes, all in accordance with the AATCC Test Method noted above.

Due to the multifiber component of the textile, fabric, or film, the combination of silver plated application to the nylon fibers does not exhibit any undesirable effects on the hand or drape of the target fabric itself. The metal finish is a solid finish on the nylon, such a finish which is noticeable by touch. Additionally, instrumental analysis can detect the actual conductivity of the fabric. In one embodiment an ohm meter can be used. The electrolessly silver plating enhances the feeling of the fabric and provides the desirable characteristics noted above.

Furthermore, the inventive dual purpose fabric, textile or film exhibits appreciable electrical conductivity (due to the 100% coating of the silver coated nylon fibers which are present throughout the target substrate) as measured by attaching a two-inch by two-inch fabric specimen to two electrodes and applying a voltage gradient of about 100 volts per inch through the fabric (i.e., in accordance with AATCC Test Method 76-1978). The measured resistance in ohms per square inch should be a minimum of 10 in order to provide a substantially electrically conductive fabric.

Nowhere within the prior art has such a specific treated dual purpose fabric, textile or film or method of making thereof been disclosed, utilized, or fairly suggested. Others have produced a product (marketed under the trade name X-STATIC®) which is a nylon yarn electrolessly plated with a silver coating on the 100% of the nylon yarn. This yarn is highly electrically conductive and can be utilized for static charge dissipation but cannot be dyed.

Any combinations of polyamides, such as nylon 6 and nylon 6.6, can be used with other fibers. Natural or synthetic fiber may be utilized as the base for coating the fabric, textile or film with silver as long as the other fiber can be dyed so the resulting substrate can hold color. Thus, natural (cotton, wool, and the like) or synthetic fibers (polyester, polyolefin, spandex and the like) may constitute one part of the target substrate, either by itself or in any combinations or mixtures. Synthetics, naturals, or blends or combinations thereof can be used. One embodiment uses at least 5% polyamides, such as nylon 6 and nylon 6.6, as the silver coated fiber. As for the other yarn types, for instance, and without intending any limitations therein, polyolefins, such as polyethylene, polypropylene, and polybutylene, halogenated polymers, such as polyvinyl chloride, polyesters, such as polyethylene terephthalate, polyester/polyethers, polyurethane, as well as spandex/elastane (block copolymer of polyurethane and polyethylene glycol) homopolymers, copolymers, or terpolymers in any combination of such monomers, and the like, may be utilized within this invention. In one embodiment polyethylene terephthalate (a polyester) can be used as the other fiber in the substrate.

Additionally, the target fabric may be coated with any number of different films, including those listed in greater detail below. As mentioned previously, the invention allows the non silver coated fibers in the substrate to be dyed or colored to provide other aesthetic features for the end user with any type of colorant, such as, for example, poly(oxyalkylenated) colorants, as well as pigments, dyes, tints, and the like. Other additives may also be present on and/or within the target fabric or yarn, including antistatic agents, brightening compounds, nucleating agents, antioxidants, UV stabilizers, fillers, permanent press finishes, softeners, lubricants, curing accelerators, and the like. Other optional and supplemental finishes to the inventive fabrics can be employed such as soil release agents, which improve the wettability and washability of the fabric. Other finishes such as soil release agents may be employed and include those which provide hydrophilicity of the surface of polyester. With such a modified surface, again, the fabric imparts improved comfort to a wearer by wicking moisture. Additionally, other potential additives and/or finishes may include water repellent fluorocarbons and their derivatives.

In an embodiment treatment comprises at least 2%, alternatively 3%, or alternatively 5% of polyamide material such as nylon 6 or nylon 6.6 so that this component of the substrate can be electrolessly plated with metal. Silver provides good overall desired characteristics, such as, good antimicrobial and/or odor reducing characteristics, and, most importantly, wash durability on the target substrate as well as electrical and thermal conductivity. It is contemplated that other metals may also be suitable for their desired characteristics.

The term silver particle is intended to encompass any compound which comprises of silver in its elemental or ionic state (thus Ag° or Ag+ may be present). Metal salts may also be present in some amount either in a pure state, or reduced to produce the desired metal particles.

The selected substrate can be a combination of a polyamide yarn such as nylon 6 or nylon 6.6 and other fibers, or a fabric comprising individual fibers or yarns (with the ability of the polyamide fibers to be electrolessly silver plated). The non polyamide individual fibers or yarns may be of any typical source for utilization within fabrics, including natural fibers (cotton, wool, ramie, hemp, linen, and the like), synthetic fibers (polyolefins, polyesters, polyaramids, acetates, rayon, acrylics, spandex/elastane and the like), and inorganic fibers (fiberglass, boron fibers, and the like). The target yarn may be of any denier, may be of multi- or mono-filament, may be false-twisted or twisted, or may incorporate multiple denier fibers or filaments into one single yarn through twisting, melting, spun and the like. The target fabrics may be produced of the same types of yarns discussed above, including any blends thereof. Such fabrics may be of any standard construction, including knit, woven, or non-woven forms.

The inventive fabrics may also be utilized in any suitable application, including, without limitation, apparel, upholstery, bedding, wiping cloths, towels, gloves, rugs, floor mats, drapery, napery, bar runners, textile bags, awnings, vehicle covers, boat covers, tents, and any similar applications.

The polyamide/other fiber substrates can be electrolessly silver plated either before or after the fabric is dyed.

### DESCRIPTION OF THE PREFERRED EMBODIMENTS

Examples of products within the scope of the present invention are set forth below.

Silver coated nylon and another non-coated fiber (e.g. polyester), were warp knitted together so that the nylon fibers were on one side of the fabric and the polyester fibers were on the other. The fabric was electrolessly silver coated and the result was a fabric that was silver coated on one side, for example on the nylon fiber side, and the other side was untouched by the silver and retained its color for dying at a later stage. An additional example shows that silver coated yarn can be knit with non-silver coated yarn so that the silver yarn is predominantly on one side of the fabric and the other side is comprised of predominantly the non-silver coated yarn, which can be dyed after the garment has been made.

### Example 1

A warp knit fabric comprised of polyester filament yarn on one side and nylon filament yarn on the other was constructed. The double-sided fabric construction was used so that the polyester showed predominately on one side of the fabric and the nylon yarn showed predominantly on the other side of the fabric. The fabric weight was approximately 3.6 ounces per square yard before the silver plating process and comprised by weight of approximately 50% polyester and 50% nylon. A fabric was used, that had already been disperse dyed, prior to electrolessly silver plating the nylon in the fabric. The dyed fabric was processed using an electrolessly silver plating process that deposited approximately 17% silver onto the nylon fibers. Prior to electrolessly plating the fabric, the garment was scoured and then chemically treated to sensitize the nylon fibers and enhance the plating process. The fabric was washed thoroughly after the plating to remove any residual chemicals from the process. Once washed, the fabric was dried and rolled onto a tube for further processing.

This fabric was cut and sewn into tee shirts, suitable for athletes. The tee-shirts were cut in such a manner that allowed the dyed side of the double sided fabric to be on the outside of the garment and the silver coated nylon side to be on the inside of the garment.

### Example 2

An alternative method of production was also employed for another garment. A seamless tank top garment was produced using two types of yarn. One yarn, a 70 denier nylon yarn, was knitted to the outside of the garment. The other yarn was a covered yarn” comprised of 30 denier silver coated nylon yarn which was wrapped around a core yarn of 20 denier spandex. This covered yarn was knitted on the inside of the garment. Once the garment was knitted into a blank it was cut and seamed to produce the final garment. The garment was dyed and due to the knitting process employed, the silver coated nylon covered yarn was predominately on the inside of the garment. This method of manufacture produced a garment that has the silver on the inside of the fabric, or in other words against the wearer's skin. The garment was then dyed to the desired color. The durable silver coated yarn not only retains its integrity over the target fabric surface, but also continues to provide effective antimicrobial treatment, thermal properties and electrical conductive properties as well.

In one embodiment, the invention relates to a two sided fabric that comprises a first side and a second side, wherein the first side comprises nylon fibers coated with silver and the second side being a non nylon fiber that has not been coated with silver. In a variation, three or more fiber types can be used in one fabric.

In a variation of this embodiment, the second side is a filament polyester yarn or a spun polyester yarn or spun polyester and cotton yarn blend.

In a further variation, the first side cannot be dyed with color using normal dye procedures. By normal dye procedures it is meant disperse dyeing and/or acid dyeing. In a further variation, the second side can be dyed using normal dye procedures.

In an embodiment, the fabric is very durable so that silver is integrally retained with the nylon fibers so that after 30 washes, as performed in accordance with wash procedure of AATCC test method 130-1981, at least 65% of the silver on the first side is retained relative to an amount of silver on the first side prior to any wash. In a variation, at least 75% of the silver on the first side is retained relative to an amount of silver on the first side prior to any wash under the same conditions. In a further variation, at least 85% of the silver on the first side is retained relative to an amount of silver on the first side prior to any wash.

In an alternate embodiment, the first side with the silver coated fiber is electrically conductive. In a variation of this embodiment, the silver is present in an amount that allows between 1 amp of current at 24 volts to 100 amps of current at 24 volts over one square meter of fabric. In a variation, the silver may be present in an amount that allows between 10 amps of current at 24 volts to 75 amps of current at 24 volts over one square meter of fabric. The silver may be present in parallel of in series to attain this current at the specified voltage.

In one embodiment, the silver on said first side is applied electrolessly. In a variation, the silver may be applied electrolytically.

In a variation of an embodiment, the said first side of the fabric is thermally more conductive than the second side.

In one embodiment, the fabric on one side is 100% nylon fabric.

In an alternate embodiment, the invention relates to a method of manufacturing a two sided fabric that is silver coated on a first side and not silver coated on a second side, wherein the method comprises using a method selected from the group consisting of a non-woven method, a warp knit method, a circular knit method and a woven method to produce a base fabric that has a nylon fiber on the first side and a non-nylon fiber on the second side wherein the base fabric is then processed by electrolessly silver plating the base fabric so that to produce the two sided fabric that has nylon fibers coated with silver on the first side and non nylon fibers without a silver coating on the second side.

In a variation of the method, the base fabric can be dyed prior to the electrolessly silver plating step. Alternatively, the fabric can also be dyed if an electrolytic silver plating step is used.

In an embodiment, the first side of the two sided fabric exhibits a log kill rate for Staphylococcus aureus of at least 1.5 and a log kill rate for Klebsiella pneumoniae of at least 1.5 after 30 washes as performed in accordance with wash procedure AATCC Test Method 130-1981.

In an embodiment, the first side has between about 8% by weight silver and 35% by weight silver. Alternatively, the first side has between about 15 and 25% by weight silver, or alternatively between about 18 and 22% silver.

The fabric of the invention can be used for any of a plurality of items. For example, the fabric can be used in any athletic gear, including tee-shirts, shorts and sweatpants. It is also easily applicable to pants or dresses, for socks, for nightwear such as nightgowns and/or pajamas, for bed sheets and/or pillowcases, for shorts, for underwear, for undergarments such as bras, or the fabric can be used in any of a plurality of other items. The fabric can also be used as a medical garment.

In an alternate embodiment, the fabric can be used to alleviate symptoms from shingles. In a variation, the method of alleviating pain symptoms from shingles in an individual comprises wearing an article of clothing comprising a two sided fabric with an inside and an outside, the inside of the fabric contacts the individual and comprises a nylon fiber coated with silver, the outside does not contact the individual and comprises a non-nylon fiber without a silver coating.

In an alternate embodiment, the fabric can be used to alleviate symptoms on a burn victim. In a variation, the method of alleviating pain symptoms from burns in an individual

What is claimed is:

1. A two sided fabric that comprises a first side and a second side, wherein the first side comprises nylon fibers coated with silver and the second side being a non nylon fiber that has not been coated with silver.

2. The fabric of claim 1, wherein the said second side is a filament polyester or a spun polyester or spun polyester and cotton.

3. The fabric of claim 1, wherein three or more fibers are used in the fabric.

4. The fabric of claim 1, wherein the second side can be dyed by disperse dyeing and/or acid dyeing.

5. The fabric of claim 1, wherein the silver is integrally retained to the nylon fibers so that after 30 washes, as performed in accordance with wash procedure of AATCC test method 130-1981, at least 65% of the silver on the first side is retained relative to an amount of silver on the first side prior to any wash.

6. The fabric of claim 1, wherein the first side with the silver coated fiber is electrically conductive and can conduct between 1 amp of current at 24 volts to 100 amps of current at 24 volts over one square meter of fabric.

7. The fabric of claim 1, wherein the silver on said first side is applied electrolessly.

8. The Fabric of claim 1, wherein the said first side is thermally more conductive than the second side.

9. A method of manufacturing a two sided fabric that is silver coated on a first side and not silver coated on a second side, wherein the method comprises using a method selected from the group consisting of a non-woven method, a warp knit method, a circular knit method and a woven method to produce a base fabric that has a nylon fiber on the first side and a non-nylon fiber on the second side wherein the base fabric is then processed by electrolessly silver plating the base fabric so that to produce the two sided fabric that has nylon fibers coated with silver on the first side and non nylon fibers without a silver coating on the second side.

10. The method of claim 8, wherein the base fabric has been dyed prior to the electrolessly silver plating step.

11. The two sided fabric of claim 1, wherein the first side of the two sided fabric exhibits a log kill rate for Staphylococcus aureus of at least 1.5 and a log kill rate for Klebsiella pneumoniae of at least 1.5 after 30 washes as performed in accordance with wash procedure AATCC Test Method 130-1981.

12. The two sided fabric of claim 1, wherein the first side has between about 8% by weight silver and 35% by weight silver.

13. A tee-shirt comprising the fabric in claim 1.

14. Nightwear comprising the two sided fabric of claim 1.

15. The nightwear of claim 14, wherein the nightwear is pajamas or a nightgown.

16. A bed sheet, or pillow case, wherein said bed sheet or pillow case comprises the two sided fabric of claim 1.

17. A pair of shorts comprising the fabric of claim 1.

18. A pair of underwear comprising the fabric of claim 1.

19. A bra comprising in part or in whole the fabric of claim 1.

20. A method of alleviating pain symptoms from shingles in an individual comprising wearing an article of clothing comprising a two sided fabric with an inside and an outside, the inside of the fabric contacts the individual and comprises a nylon fiber coated with silver, the outside does not contact the individual and comprises a non-nylon fiber without a silver coating.
